(19) United States
(12) Patent Application Publication
(10) Pub. No.: US 2025/0186351 A1
(43) Pub. Date: Jun. 12, 2025

(54) CO-PROCESSED EXCIPIENT COMPOSITION

(71) Applicant: ISP INVESTMENTS LLC, Wilmington, DE (US)

(72) Inventors: Brian PHILLIPS, Haddonfield, NJ (US); Quyen Vo SCHWING, Mount Laurel, NJ (US); Thomas DÜRIG, West Chester, PA (US); Joseph Christopher AMBROSI, West Chester, PA (US)

(73) Assignee: ISP INVESTMENTS LLC, Wilmington, DE (US)

(21) Appl. No.: 18/845,339

(22) PCT Filed: Mar. 6, 2023
(86) PCT No.: PCT/US2023/014616

§ 371 Date: Sep. 9, 2024

Related U.S. Application Data
(60) Provisional application No. 63/318,340, filed on Mar. 9, 2022.

(51) Int. Cl. A61K 9/20

(52) U.S. Cl. CPC A61K 9/2027; A61K 9/2009; A61K 9/2095

(57) ABSTRACT

A co-processed excipient composition comprising (i) vinyl lactam derived polymer; (ii) silica; and (iii) at least one lubricant is provided in the present disclosure. The co-processed excipient has a Brookfield flow factor of 5 to 9. Also provided a process for preparing the same. The co-processed excipient is used further in continuous or direct compression batch manufacturing with superior flow properties and self-lubrication.

### FIELD OF THE INVENTION

The presently disclosed process(es), procedure(s), method(s), product(s), result(s), and/or concept(s) (collectively referred to hereinafter as the “present disclosure”) relates generally to a co-processed excipient composition derived from a vinyl lactam-based polymer and a process for preparing the same. The present disclosure further relates to a composition(s) derived from the present co-processed excipient composition and a process(s) for preparing the same.

### BACKGROUND OF THE INVENTION

Tablets and capsules represent the most preferred and most commonly dispensed pharmaceutical dosage forms for administering active pharmaceutical ingredients (APIs). Tablets can be manufactured by direct compression or via dry, wet or melt granulation of drug(s)/excipient(s) mixtures. Direct compression continues being the most preferred manufacturing process to produce tablets due to some advantages such as time saving, ease of production, absence of heat and moisture in the process, and the like. The choice of tableting process is highly influenced by the flowability and compressibility of tableting mixture, for example, active pharmaceutical ingredient (API)-excipient mixture. Two major factors which disparagingly affect the direct compression process are: compressibility and flowability of the tableting mixture. Direct compression method demands excellent flowability and compression of the tableting mixture. Most of the commercially available excipients fail to meet the desired set of functionalities as this is not an easy task to achieve as the more compressible a material is, the less flowable it will be.

One of the techniques to enhance certain functionalities of an existing excipient is using spray drying or granulation method. WO2012/116402A1 assigned to University of Monash teaches binder powders for use in powder material processing. These binder powders are prepared by using techniques such as spray drying and mechanofusion. These processes however lead to reduction in particle size of the polymer. Further, these processes are costly, time consuming, and require specialized instruments that are not commonly available at manufacturing units.

Another important technique is co-processing. The co-processing is termed as a science of particle engineering wherein two or more excipients are synergistically combined into a single multifunctional excipient with superior intrinsic performance.

U.S. Pat. No. 4,734,285 assigned to Dow Chemical Company teaches delayed release solid tablets of a therapeutically active composition and a process to prepare such a composition. Fine particles, which can pass through a 100-mesh screen (149 micrometer mesh size) and preferably 140-mesh screen (105 micrometer mesh size), of hydroxypropyl methylcellulose ether are present as an excipient in the tablet composition. These fine particles are very small in size and shows poor flow properties. Poor particle flow can lead to consolidation of the powder bed in processing equipment, such as storage bins and tablet press feed hoppers. Problems can include increased inconsistency in tablet weight or tablet crushing strength from tablet-to-tablet as well as inconsistency in the amount of active ingredients incorporated into each dosage form.

WO2004/022601 assigned to JRS Pharma LP and U.S. Pat. No. 5,585,115 assigned to Edward H. Mendell Co., Inc. teach an agglomerated microcrystalline cellulose blend containing silicon dioxide, purported to have improved compressibility. These disclosures state that silicon dioxide is a critical component to improve compressibility. The two-step process described includes spray granulation followed by wet granulation. The prepared granules are further dried using heat, which is not advantageous. Further, the granulation is time consuming and adds cost to the process due to the time lost, additional labor, energy consumption and additional equipment required.

Several processes for drying-grinding moist cellulose derivatives are known in the art, such as described in the patent applications GB 2262527A; EP 0824107 A2; EP-B 0370447 (equivalent to U.S. Pat. No. 4,979,681); EP 1127895 A1 (equivalent to U.S. Pat. No. 6,509,461); EP 0954536 A1 (equivalent to U.S. Pat. No. 6,320,043); WO96/00748 A1; WO2011/046679 (equivalent to US 2012/187225) and WO2012/138532.

Further, US2012/160944A1 assigned to ICEUTICA PTY LTD teaches a method to produce nano and micro-particle powders of a biologically active material which have improved powder handling properties using dry milling process.

US2012/0178822A assigned to ISP INVESTMENTS INC teaches coprocessing of PVP and calcium silicate by using ball milling, spray drying or freeze drying.

The increase in flow of cellulose polymers by co-milling microcrystalline cellulose with nano-silica is described in J. Pharm. Sci. 2011 November; 100(11):4943-52, Chattoraj S, Shi L, Sun CC.

Further, U.S. Pat. No. 10,596,261 assigned to Hercules LLC teaches a co-processed excipient having vinyl lactam derived polymers and deagglomerated silica as a co-processing agent. The vinyl lactam derived polymer and deagglomerated silica are co-processed together in a continuous manner to obtain a co-processed excipient. The co-processed excipient has a Brookfield cohesion factor of less than 0.2 kPa and a bulk density of at least 0.249 g/ml.

Similarly, U.S. Pat. No. 10,172,944 assigned to Hercules LLC teaches a co-processed excipient composition having a cellulose derived polymer and a deagglomerated silica as a co-processing agent. The co-processed excipient is prepared in a continuous process and has a Brookfield cohesion factor of less than 0.2 kPa and a bulk density of at least 0.249 g/ml.

Cross-linked polyvinyl pyrrolidone (crospovidone type B) is a commonly used excipient in oral solid dosage (OSD) forms which encourages rapid disintegration. Further, agglomerated silica is a glidant commonly used to enhance flow of OSD blends. Another excipient which is commonly used in the OSD form is lubricant. The lubricants are known to extend the lifetime of tooling used during OSD manufacturing. Nevertheless, these lubricants are notoriously difficult to feed due to their low bulk densities and cohesion, making their implementation in continuous manufacturing schemes problematic; these challenges will worsen as the pharmaceutical industry increasingly adopts continuous manufacturing process.

Therefore, there is a long-felt need in the art to develop a co-processed excipient composition having superior or excellent flow properties and compression behavior wherein the excipient when used further in pharmaceutical formulations, particularly OSD forms, provide OSD with superior hardness.

### SUMMARY OF THE INVENTION

In one aspect, the present disclosure provides a co-processed excipient comprising (i) from 90.0 wt. % to 99.9 wt. % of a vinyl lactam derived polymer comprising a monomer selected from the group consisting of N-vinyl-2-pyrrolidone, N-vinyl-2-caprolactam, N-vinyl-3-methyl-2-pyrrolidone, N-vinyl-3-methyl-2-caprolactam, N-vinyl-4-methyl-2-pyrrolidone, N-vinyl-4-methyl-2-caprolactam, N-vinyl-5-methyl-2-pyrrolidone, N-vinyl-5,5-dimethyl-2-pyrrolidone, N-vinyl-3,3,5-trimethyl-2-pyrrolidone, N-vinyl-5-methyl-5-ethyl-2-pyrrolidone, N-vinyl-3,4,5-trimethyl-3-ethyl-2-pyrrolidone, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, and combinations thereof; (ii) from 0.1 wt. % to 5.0 wt. % of silica; and (iii) from 0.1 wt. % to 5.0 wt. % of at least one lubricant. In one non-limiting embodiment of the present disclosure, the vinyl lactam derived polymer is selected from the group consisting of poly(vinyl pyrrolidone), cross-linked poly(vinyl pyrrolidone), and combinations thereof.

In one non-limiting embodiment of the present disclosure, the vinyl lactam derived polymer is present in an amount of 98.0 wt. % to 99.0 wt. %, based on the total weight of the excipient composition. In another non-limiting embodiment of the present disclosure, the silica is present in an amount of 0.5 wt. % to 2.0 wt. %, based on the total weight of the excipient composition. In a still another non-limiting embodiment of the present disclosure, the lubricant is present in an amount of 0.1 wt. % to 3.0 wt. %, based on the total weight of the excipient composition.

In one non-limiting embodiment of the present disclosure, the silica is selected from the group consisting of colloidal silica, fumed silica, a silicon dioxide, a calcium silicate and any combinations thereof. In a still another non-limiting embodiment of the present disclosure, the lubricant is selected from the group consisting of sodium stearyl fumarate, magnesium stearate, stearic acid, glyceryl dibehenate, and any combinations thereof.

In one non-limiting embodiment of the present disclosure, the co-processed excipient has a Brookfield flow factor of 5 to 9.

Another aspect of the present disclosure provides a process for preparing the co-processed excipient of the present disclosure, wherein the process comprises the steps of: (i) blending a vinyl lactam derived polymer, silica and a lubricant to obtain a blend, and (ii) milling the resultant blend to obtain a co-processed excipient. In one non-limiting embodiment of the present disclosure, the process is a single step process or a two-step process. In another non-limiting embodiment of the present disclosure, the process is a two-step process comprising the steps of: (i) blending a vinyl lactam derived polymer and silica; (ii) adding a lubricant to the blend of step (i) to obtain a blend; and (iii) milling the resultant blend of step (ii) to obtain a co-processed excipient.

In a still another aspect, the present disclosure provides a composition comprising the co-processed excipient of the present disclosure for use in an industrial application selected from paints and coatings, personal care, detergents, pharmaceuticals, nutraceuticals, ceramics, insulators, pet food, animal food and human food, agricultural products, adhesives, electroplating, inks, dyes, paper, catalytic convertors, and electronics. In one non-limiting embodiment of the present disclosure, the composition is used in pharmaceuticals.

In yet another aspect, the present disclosure provides a directly compressible pharmaceutical composition comprising: (i) an active pharmaceutical ingredient; (ii) the co-processed excipient of the present disclosure; and (iii) optionally one or more pharmaceutically acceptable additives.

In another aspect, the present disclosure provides a process of preparing the directly compressible pharmaceutical composition of the present disclosure comprising the steps of: (i) blending the active pharmaceutical ingredient, the co-processed excipient of the present disclosure, and optionally one or more additives; and (ii) compressing the resulting mixture of step (i).

### BRIEF DESCRIPTION OF THE FIGURES AND DRAWINGS

Objects, features, and advantages of the present disclosure will become apparent upon reading the following description in conjunction with the drawings/figures, in which:

FIG. 1 shows Brookfield flow function coefficient (fcc) of the co-processed excipient of Example 1 prepared by using cross-linked polyvinylpyrrolidone (PVPP) and 0.7 wt. %, 1.0 wt. % and 1.4 wt. % of silica (with-out adding any lubricants).

FIG. 2 shows the Feed Factor (Loss-in-weight feeder (LWF)) throughput analysis of the present co-processed excipient composition of Example 2 (Ex.2) and its comparison with the LWF throughput analysis of PVPP alone.

FIG. 3 shows Brookfield flow function coefficient of the present co-processed excipient composition (Ex.2) and its comparison with PVPP alone.

FIG. 4 shows Feed Factor (Loss-in-weight feeder (LWF) throughput analysis) of the present co-processed excipient composition of Example 3 prepared by using different lubricants.

FIG. 5 shows crushing strength or hardness of the co-processed excipient compositions of Example 3 and its comparison with the co-processed excipient composition of Example 1 (Ex.1A).

FIG. 6 shows hardness (kp) of directly compressed tablet samples of Ex.3B and Ex.3E.

FIG. 7 shows Feed Factor (Loss-in-weight feeder (LWF) throughput analysis of the present co-processed excipient composition of Example 4 and Example 3 (Ex.3B) prepared by using 3.0 wt. % and 1.0 wt. % of sodium stearyl fumarate.

FIG. 8 shows ejection force data of Acetaminophen tablet samples prepared by using the present co-processed excipient of Example 5A and its comparison with control tablet sample CE.5A.

FIG. 9 and FIG. 10 show hardness (kp) and disintegration time (sec) of Acetaminophen tablet samples prepared by using the present co-processed excipient of Example 5A and its comparison with control tablet sample CE.5A.

FIG. 11 shows crushing strength (kP) of the present tablet composition i.e., EX.5A, EX.5B and EX.5C and its comparison with the control tablet samples CE.5A and CE.5B.

FIG. 12 shows hardness (kp) of Ibuprofen tablet samples prepared from the direct compression of the co-processed excipient compositions Ex.3B (with 1 wt. % SSF) and Ex. 4 (with 3.0 wt. % SSF).

### DESCRIPTION OF THE INVENTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is/are capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art.

All patents, published patent applications, and non-patent publications mentioned in the specification are indicative of the level of skill of those skilled in the art to which this present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, and/or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combinations of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “bulk density” refers to Bulk density (BD) defined as the ratio of apparent volume to mass of the material taken, called untapped bulk density, and also the ratio of tapped volume to mass of material taken, called tapped bulk density. A useful procedure for measuring these bulk densities is described in United States Pharmacopeia 24, Test 616 “Bulk Density and Tapped Density,” United States Pharmacopeia Convention, Inc., Rockville, Md., 1999.

As used herein, the term “deagglomeration” refers to a process of breaking up or dispersing that which has agglomerated, aggregated, or clustered together.

As used herein, the term “co-processed excipient composition” refers to a co-processed excipient which is a combination of two or more compendial or non-compendial excipients designed to physically modify their properties in a manner not achievable by simple physical mixing and without significant chemical change.

As used herein, the term “mill” refers to a high-speed hammer mill for dry grinding or deagglomerating of various products. In particular, the mill is utilized as a hammer mill, which is characterized by an impact process between the blade and input material. Material and air enter the mill and impacted from the blade; subsequently gravity and material momentum carry force the material through a screen.

As used herein, the term “blender” refers to a continuous single or double helix ribbon blender with a residence time of at least one second; or a blender for batch processing including, but not limited, to a “V” blender or a cone blender.

As used herein, the term “compaction” means a simultaneous process of compression and consolidation of a two-phase system (solid air) due to the applied force.

As used herein, the term “direct compression” or “DC” refers to obtaining a formulation by directly compressing and molding a raw material powder. This process is described in publications such as “The Theory and Practice of Industrial Pharmacy” (Third Edition) (Leon Lachman, et al.: LEA & FEBIGER 1986) and Pharmaceutical Dosage Forms: Tablets Volume 1 (Second Edition) (Herbert A. Lieberman, et al.: MARCEL DEKKER INC. 1989).

As used herein, the term “continuous process” refers to production that is not executed batch wise but steadily such as production on a continuous blend. In non-continuous processes, i.e., batch production processes, insertion of the raw materials into the machine/mill and subsequent unloading of the newly produced composition from the machine/mill occupies too much time to make low-cost production impossible. The significance of the term “continuous production” here is the implication of the advantages gained by an assembly line with each step characterized by an average residence time.

In one aspect, the present disclosure provides a co-processed excipient composition comprising (i) a vinyl lactam derived polymer; (ii) at least one silica; and (iii) at least one lubricant.

In one non-limiting embodiment of the present disclosure, the vinyl lactam derived polymer comprises monomers selected from the group consisting of N-vinyl-2-pyrrolidone, N-vinyl-2-caprolactam, N-vinyl-3-methyl-2-pyrrolidone, N-vinyl-3-methyl-2-caprolactam, N-vinyl-4-methyl-2-pyrrolidone, N-vinyl-4-methyl-2-caprolactam, N-vinyl-5-methyl-2-pyrrolidone, N-vinyl-5,5-dimethyl-2-pyrrolidone, N-vinyl-3,3,5-trimethyl-2-pyrrolidone, N-vinyl-5-methyl-5-ethyl-2-pyrrolidone, N-vinyl-3,4,5-trimethyl-3-ethyl-2-pyrrolidone, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, and any combinations thereof; In one non-limiting embodiment of the present disclosure, the vinyl lactam derived polymer can be a polyvinylpyrrolidone or cross-linked polyvinyl pyrrolidone, or combinations thereof.

The polyvinyl pyrrolidone (PVP) useful for the purpose of the present disclosure refers to a polymer available in different pharmaceutical grades. A particularly preferred source of polyvinyl pyrrolidone can be Plasdone™ Povidone marketed by Ashland LLC.

Further, the polyvinylpyrrolidone useful for the purpose of the present disclosure can be a cross-linked polyvinylpyrrolidone (also termed as cross-linked PVP or PVPP). Such polymers are also commercially available in different pharmaceutical grades and can be used for the purpose of the present disclosure. Suitable and non-limiting examples of such commercially available cross-linked PVP can include Polyplasdone XL®, Polyplasdone XL-10® (crospovidone type B), Polyplasdone INF-10, and Polyplasdone ultra as marketed by Ashland LLC. The cross-linked PVP are commonly used excipients in oral solid dosage forms which encourages rapid disintegration.

Further, the vinyl lactam derived polymer can be present in an amount of from 90.0 wt. % to 99.9 wt. %, based on the total weight of the co-processed excipient composition. In one non-limiting embodiment of the present disclosure, the amount of vinyl lactam derived polymer can vary in the range of from about 90.0 wt. % to about 95.0 wt. %, or from about 95.0 wt. % to about 99.0 wt. %, or from about 98.0 wt. % to about 99.0 wt. %, based on the total weight of the co-processed excipient composition.

The silica used in the co-processed excipient composition of the present disclosure can be selected from the group consisting of colloidal silica, fumed silica, a silicon dioxide, a calcium silicate, and any combinations thereof.

In one non-limiting embodiment of the present disclosure, the silica can be a fumed silica. Further, the silica can be present in an amount of from about 0.1 wt. % to about 5.0 wt. %, based on the total weight of the co-processed excipient. In one non-limiting embodiment of the present disclosure, the amount of silica can vary in the range of from about 0.1 wt. % to about 4.0 wt. %, or from about 0.1 wt. % to about 3.0 wt. %, or from about 0.1 wt. % to about 2.0 wt. %, or from about 0.5 wt. % to about 4.0 wt. %, or from about 0.5 wt. % to about 3.0 wt. %, or from about 0.5 wt. % to about 2.0 wt. %, or from about 0.5 wt. % to about 1.0 wt. %, based on the total weight of the co-processed excipient composition.

Silicon dioxide, particularly colloidal silicon dioxide has particles size particularly less than 500 nm, more particularly less than 400 nm. Those skilled in the art will appreciate that the name and/or method of preparation of the silicon dioxide utilized in the present invention is not determinative of the usefulness of the product. Rather, it has been surprisingly discovered that it is the physical characteristics of the silicon dioxide which are critical. In particular, it has been discovered that silicon dioxide having a relatively large particle size (and correspondingly small surface area), such as silica gel, is not useful in the present disclosure. Silica itself is a submicron, fluffy, light, loose, bluish-white, odorless and tasteless amorphous powder which is commercially available from a number of sources, including Cabot Corporation (under the tradename Cab-O-Sil); Degussa, Inc. (under the tradename Aerosil®); E.I. DuPont & Co.; and W.R. Grace & Co. Colloidal silicon dioxide is also known as colloidal silica, fumed silica, amorphous fumed silica, silicon dioxide, amorphous silica, light anhydrous silicic acid, silicic anhydride, and silicon dioxide fumed, among others. However, the amount of silicon dioxide included in pharmaceutical applications is limited and it is in the range of 0.01-1% by weight. Handbook of Pharmaceutical Excipients, COPYRGT. 1986 American Pharmaceutical Association, page 255.

The co-processed excipient of the present disclosure can further comprise at least one lubricant. The lubricants are typically added to reduce tablet ejection force. Suitable and non-limiting examples of such lubricants for the purpose of the present disclosure can include, but are not limited to, magnesium stearate, calcium stearate, sodium stearyl fumarate, stearic acid, glyceryl dibehenate, talc, sucrose fatty acid esters, and the like. In one non-limiting embodiment of the present disclosure, the lubricant can be sodium stearyl fumarate. Further, the lubricant can be added in an amount varying in the range of from about 0.1 wt. % to about 5.0 wt. %, or from about 0.1 wt. % to about 3.0 wt. %, based on the total weight of the excipient composition.

The co-processed excipient composition of the present disclosure can be prepared by co-processing various ingredients as herein above described. In another aspect, the present disclosure provides a process for preparing the co-processed excipient of the present disclosure. The process according to the present disclosure typically involves blending and milling of various ingredients. Further, the present process can be a single step process or a two-step process. In one non-limiting embodiment of the present disclosure, the process can be a two-step process comprising the steps of: (i) blending a vinyl lactam derived polymer and silica from 1 minute to 30 minutes; (ii) adding the lubricant to the blend of step (i) and blending for an additional 1 minute to 30 minutes to obtain a blend; and (iii) milling the resultant blend of step (ii) from 1 to 30 passes to obtain a co-processed excipient.

The process according to the present disclosure provides the co-processed excipient with a brookfield flow factor of 5 to 9. Further, the process according to the present disclosure is advantageous as it avoids two additional time consuming manufacturing steps i.e. separate addition of silica and lubricants.

Another aspect of the present disclosure provides a composition comprising the co-processed excipient composition of the present disclosure wherein the composition can be useful for industrial applications. Suitable and non-limiting examples of such industrial applications can include, but are not limited to, paints and coatings, personal care, detergents, pharmaceuticals, nutraceuticals, ceramics, insulators, pet food, animal food and human food, agricultural products, adhesives, electroplating, inks, dyes, paper, catalytic convertors and electronics. In one non-limiting embodiment of the present disclosure, the composition can be used in pharmaceutical applications.

In still another aspect, the present disclosure provides a pharmaceutical composition comprising the co-processed excipient of the present disclosure. The pharmaceutical composition according to the present disclosure can comprise (i) an active pharmaceutical ingredient; and (ii) the co-processed excipient of the present disclosure.

Any active pharmaceutical ingredients (API) which are well known to persons skilled in the related art can suitably be used for the purpose of the present disclosure, for example, drugs or a bio-functional ingredient(s). Suitable examples of the bio-active ingredients useful for the purpose of the present disclosure can include, but are not limited to, dietary supplements including, but not limiting to, vitamins, such as, Vitamin C, Vitamin B1, B2, B3, B6 and B12; minerals, such as, zinc, magnesium, iron, and melatonin; herbal dietary supplements, such as, curcumin, ashwagandha, and fenugreek extract; amino acids, such as, isoleucine, glycine, L-tryptophan, glucosamine, chondroitin, and the like.

In another non-limiting embodiment of the present disclosure, the active pharmaceutical ingredient can be a drug. Any drugs having a wide range of water solubilities can suitably be used in the present pharmaceutical composition. In one non-limiting embodiment of the present disclosure, the drug can be selected from the group of drugs belonging to different therapeutic classes such as antipyretic, analgesic and anti-inflammatory drugs, anthelmintic drugs, cardiovascular drugs, antibacterial drugs, bronchodilating drugs, anti-asthmatic drugs, gastrointestinal drugs, antidiabetic drugs, antiprotozoal drugs, antiviral drugs, anti-epileptic drugs, anti-diuretic drugs, or its pharmaceutically acceptable salts and esters thereof.

Further, the active pharmaceutical ingredient can be present in pharmaceutical effective amount. The amount of the active pharmaceutical ingredient can be varied depending upon various factors including, but not limiting to, type of drug or drugs being used, nature and severity of the ailment being treated/cured, the type and content of active ingredients or other ingredients contained in the pharmaceutical composition, dosage form, patient's age, weight, health condition and eating behavior, drug administration time, and the like. In one non-limiting embodiment of the present disclosure, the amount of active pharmaceutical ingredient can vary in the range of from about 10.0 wt. % to about 70.0 wt. %, or from about 20.0 wt. % to about 70.0 wt. %, of the total weight of the pharmaceutical composition.

The composition for pharmaceutical application according to the present disclosure can further comprise at least one pharmaceutical acceptable excipient. The pharmaceutical acceptable excipients which are commonly used in the pharmaceutical compositions are also suitable for use in the present composition, for example, excipients as described in Handbook of Pharmaceutical Excipient, Rows et al., Eds., 4th Edition, Pharmaceutical Press (2003) or Remington; The Science and Practice of Pharmacy (formerly called Remington's Pharmaceutical Sciences), Alfonso R. Gennaro, ed., Lippincott Williams & Wilkins; 20th edition (Dec. 15, 2000). Examples of such excipients can include, but are not limited to, fillers, pigments, binders, lubricants, flow aids, flavors, sweeteners, preservatives, stabilizers, antioxidants, and the like.

Further, the pharmaceutical ingredient can be present in an amount without affecting the therapeutic properties of the present composition for pharmaceutical application. The pharmaceutical acceptable ingredient can be present in an amount of from about 1.0 wt. % to about 85.0 wt. %, of the total weight of the composition. In one non-limiting embodiment of the present disclosure, the amount of pharmaceutical acceptable ingredient can vary in the range of from about 5.0 wt. % to about 75.0 wt. %, or from about 5.0 wt. % to about 60.0 wt. %, of the total weight of the pharmaceutical composition.

Further, in one non-limiting embodiment of the present disclosure, the co-processed excipient of the present disclosure can be present in an amount of from about 2.0.wt. % to about 20.0 wt. %, based on the total weight of the pharmaceutical composition.

The composition for pharmaceutical application according to the present disclosure can be present in a dry solid dosage form. The dry solid dosage form are useful for delivering an accurate dosage to specific site, usually orally, but can also be administered via other routes that are known to a person skilled in the pertinent art, such as, sublingual/buccal, rectal, vaginal and ocular. In one non-limiting embodiment of the present disclosure, the composition for pharmaceutical application can be present in solid dosage forms suitable for oral administration. Such dosage forms can include, but are not limited to, tablets, capsules, powder, granules, sachets or lozenges. In one non-limiting embodiment of the present disclosure, the composition for pharmaceutical application can be tablet formulations.

The tablet formulations according to the present disclosure can be prepared by tableting methods which are well known to a person skilled in the pharmaceutical art, such as, wet granule tableting method or a dry granule tableting method or a dry direct tableting method. In one non-limiting embodiment of the present disclosure, the tablet formulations can be prepared by dry direct tablet method or a direct compression (DC) method.

In another aspect, the present disclosure provides a directly compressible tablet formulation. The directly compressible tablet formulation typically comprises: (i) the co-processed excipient of the present disclosure; (ii) at least one active pharmaceutical ingredient (API); and (iii) at least one active pharmaceutical additive.

In still another aspect, the present disclosure provides a process for preparing the directly compressible tablet formulation. The direct compressible tablet formulation can be prepared continuously or in a batch-wise manner. The process can comprise the steps of: (i) pre-milling or sieving various ingredients of the present composition such as the co-processed excipient of the present disclosure; the active pharmaceutical ingredient such as a drug(s) and the like; and the pharmaceutical acceptable additive(s) to obtain fine powdered ingredients; (ii) uniformly blending or mixing the fine powdered ingredients to obtain a homogenous blend thereof; and (iii) compressing the homogeneous blend to obtain directly compressible tablet formulation.

The co-processed excipient composition according to the present disclosure is a multifunctional disintegrant suitable for use in oral solid dosage form manufacturing. The co-processed excipient demonstrates superior flow properties and self-lubrication. The present co-processed excipient when used further in direct compression tablet manufacturing, provides tablets with improved hardness, improved process throughput and enhanced quality performance.

Further, certain aspects of the present application are illustrated in detail by way of the following examples. The examples are given herein for illustration of the application and are not intended to be limiting thereof.

### EXAMPLES

### Test Methods:

Unless indicated otherwise, the following test methods were utilized in the Examples that follows:

### Bulk Density

Bulk density test was done via standard USP method. The material is dispensed within 100 ml graduated cylinder and mass is recorded.

### Particle Size

A Malvern Mastersizer 2000 was utilized for particle-size analysis. This experimental method is called dynamic light scattering (dls)

### Flow Properties

The flow properties were measured by using a Loss-in-weight feeder (LWF). In a typical experiment, ˜1.5 kg of material was introduced to a K-TRON KSU-II LWF outfitted with a concave screw. Material was then studied as it flows at a nominal mass flowrate setpoint of ˜2 kg/hr.

The mass flow rate and CMD % (motor velocity) was then recorded over a period of time. The experiment was concluded to be over with when 5 or more values of 110% CMD are recorded (this indicates that the feeder can no longer reliably maintain mass flowrate setpoint).

The mass flow rate was then divided by the CMD %, producing a number called the feed factor. It represents the inherent flowability of the powder as it is used on the LWF. For instance, a higher feed factor indicates higher inherent flowability (i.e. the feeder required less CMD % (energy) to maintain the mass flow rate setpoint).

For bench-top flow study, Brookfield flow function tests are executed which produce the flow function coefficient (ffc). The ffc is merely 1/slope reported of the test.

### Example 1: Co-Processing of Cross-Linked Polyvinyl Pyrrolidone (PVPP) with Silica

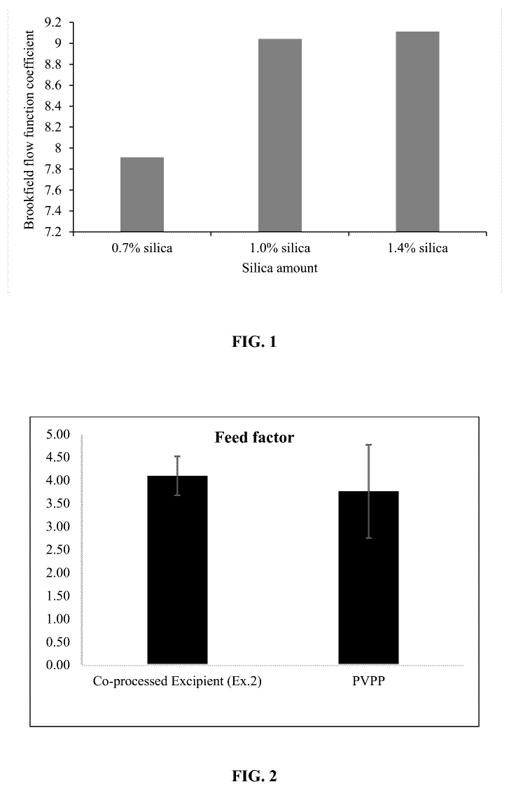

In this example, cross-linked polyvinylpyrrolidone (PVPP) (available as Polyplasdone XL-10 from Ashland LLC.) was co-processed with fumed silica (available as Cab-O-Sil from Cabot Corporation) with out adding any lubricant. Three different samples were prepared using PVPP and fumed silica in weight proportions shown in Table 1. In a typical experiment, PVPP and silica were blended for ˜10 minutes within a ribbon blender (200 Ft3) at 1.5 rpm. The Brookfield flow function coefficient (ffc) of all the samples was measured and provided in Table 1 and further illustrated in FIG. 1.

TABLE 1
Co-processed Excipients prepared by using cross-
linked PVP and Silica (With-out lubricant)

| | Ex. 1A | Ex. 1B | Ex. 1C |
|---|---|---|---|
| Compositions | % w/w | % w/w | % w/w |
| Cross-linked PVP | 99.3 | 99.0 | 98.6 |
| % Silica | 0.7 | 1.0 | 1.4 |
| ffc | 7.91 | 9.04 | 9.11 |

### Example 2: Present Co-Processed Excipient Composition (Ex.2)

The co-processed excipient composition of this example was prepared at pilot scale. 0.35 lb (0.7 wt. %) of silica and 49.15 lb (98.3 wt. %) of cross-linked polyvinylpyrrolidone (PVPP: Polyplasdone XL-10) were blended together for 10 minutes on a 5 Ft3 Ross Ribbon blender at 49.3 m/s. The obtained blend was then mixed with 0.5 lb (1.0 wt. %) of sodium stearyl fumarate (SSF) for two additional minutes. The resultant blend thus obtained was then milled using a Fitz Mill (Model JT, SN 1408) on a 0.033″ screen to obtain resultant co-processed excipient (Ex.2) (Polyplasdone XL-10 DC). The Loss-in-weight feeder (LWF) throughput analysis, Brookfield flow function coefficient (ffc) and particle size distribution (PSD) of the co-processed excipient composition (Ex.2) was measured and compared with the cross-linked PVP alone.

TABLE 2
Particle Size Distribution of co-processed excipient
composition (Ex. 2) and cross-linked PVP

| | D10 | D50 | D90 |
|---|---|---|---|
| Compositions | (μm) | (μm) | (μm) |
| Cross-linked PVP | 9.2 | 22.4 | 49.6 |
| Ex. 2 | 10 +/− 5 | 20 +/− 5 | 60 +/− 10 |

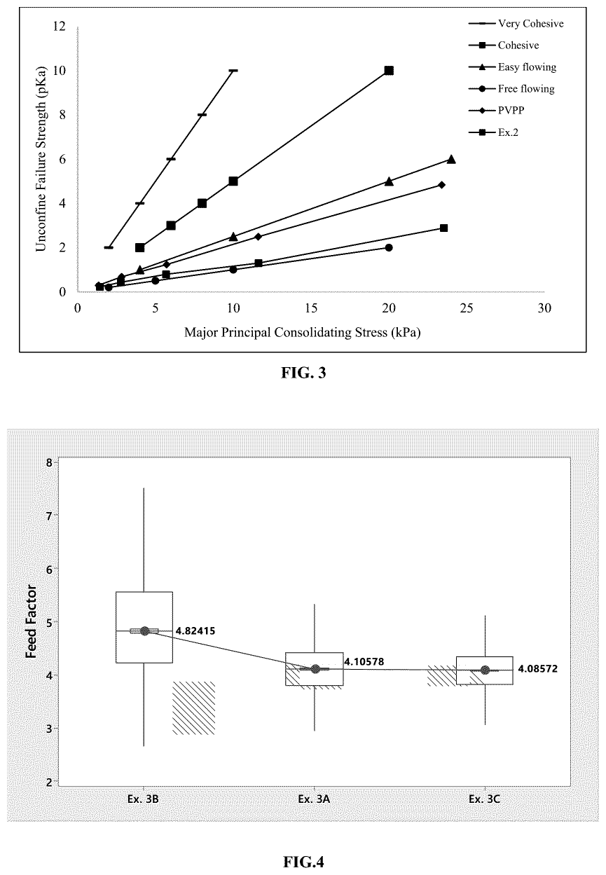

From Table 2, it is evident that the co-processing of Example 2 does not change the PSD of the co-processed excipient (Ex.2). Further, Feed Factor (the Loss-in-weight feeder (LWF)) throughput analysis and Brookfield flow function coefficient of the co-processed excipient composition (Ex.2) and its comparison with the PVPP is illustrated in FIG. 2 and FIG. 3, respectively. The provided figures clearly depict that the Feed Factor of the co-processed excipient composition (Ex.2) is increased by ˜9%, % relative standard deviation (RSD) of feed factor is decreased by 32%, and flow function increased by 29% compared to the PVPP alone.

### Example 3: Co-Processed Excipient Compositions with Different Lubricants

Three different samples (Ex.3A, Ex.3B & Ex.3C) of the present co-processed excipient compositions using different lubricants were prepared in this example. Various ingredients including their weight proportions used for preparing the co-processed excipient compositions are shown in Table 3. These excipients were prepared at lab scale. In a typical experiment, 14.0 gm (0.7 wt. %) of silica and 1966.0 gm (98.3 wt. %) of cross-linked polyvinylpyrrolidone (PVPP) were blended together for ˜10 minutes within a ribbon blender (200 Ft3) at 1.5 rpm. The obtained blend was then mixed with 20.0 gm (1.0 wt. %) of sodium stearyl fumarate (SSF) and blended for ˜5 minutes. The resultant blend thus obtained was then milled for ˜1 min using a Fitz Mill (Model DAS06) at a tip speed of 59.2 m/s, hammers forward through a 0.033″ screen to obtain a resultant co-processed excipient (Ex.3A). The feed factor analysis of all the samples of this example is illustrated in FIG. 4.

TABLE 3
Co-processed Excipients with different
lubricants and without lubricant

| | Ex. | Ex. | Ex. | Ex. | Ex. | Ex. |
|---|---|---|---|---|---|---|
| Compositions | 3A | 3B | 3C | 3D | 3E | 1A |
| Cross-linked PVP | 98.3 | 98.3 | 98.3 | 98.3 | 100.0 | 99.3 |
| Silica | 0.7 | 0.7 | 0.7 | 0.7 | Nil | 0.7 |
| Magnesium Stearyl | 1.0 | Nil | Nil | Nil | Nil | Nil |
| Fumarate | | | | | | |
| Sodium Stearyl | Nil | 1.0 | Nil | Nil | Nil | Nil |
| Fumarate | | | | | | |
| Glyceryl dibehenate | Nil | Nil | 1.0 | Nil | Nil | Nil |
| Stearic acid | Nil | Nil | Nil | 1.0 | Nil | Nil |

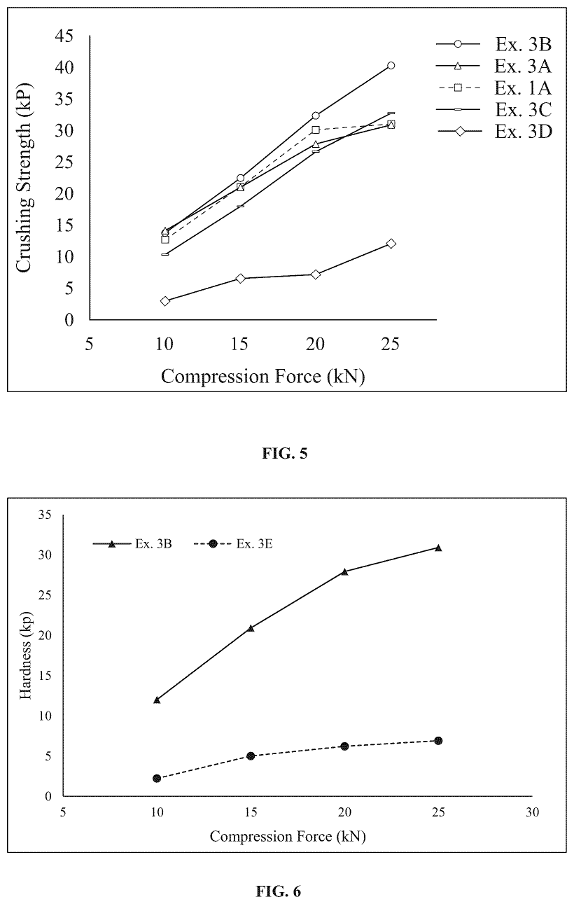

The co-processed excipient compositions (Ex.3A, Ex. 3B, Ex.3C and Ex. 3D) were further directly compressed into tablets; Cross-linked PVP (polyplasdone) alone (Ex.3E) was also directly compressed. In a typical process, the individual co-processed excipient composition was blended on a Turbula mixer for ˜10 min. The resultant homogenous blend was then compressed on a StylCam, a compaction simulator, simulating a Manesty Betapress operating at ˜37 RPM, to tablets with an individual tablet weight of 400 mg, 11.28 mm flat faced tooling (TSM B) was used at compression forces reported. These tablets were characterized for crushing strength or hardness (kp). Their crushing strength or hardness was further compared with the tablets prepared with the co-processed excipient composition of Example 1 (Ex.1A). Their comparative analysis is further illustrated in FIG. 5. An additional comparative analysis was conducted where tablet hardness of Ex. 3B was compared to Ex. 3E (FIG. 6) This demonstrated that a novel excipient comprised of co-processed cross-linked PVP, silica, and sodium stearyl fumarate (Ex. 3B) was more compressible than the original polymer (cross-linked PVP) alone (Ex. 3E).

### Example 4: Co-Processed Excipients with Different Wt. % of Sodium Stearyl Fumarate

The co-processed excipient composition of this example (Ex.4) was prepared in the same manner as described in Example 3, except 3 wt. % of the sodium stearyl fumarate was used. The feed factor analysis of this excipient composition and its comparison with the excipient composition of Example 3 (Ex. 3B with 1 wt. % SSF) is illustrated in FIG. 7.

### Example 5: Directly Compressed (DC) Acetaminophen (APAP) Tablet (Ex.5A, Ex.5B and Ex.5C)

Acetaminophen (APAP) was used as a model drug for preparing directly compressible tablets. A typical tablet formula is shown in Table 4. In a typical process, ingredients in weight proportions as listed in Table 4 were blended on a Turbula mixer for ˜10 min. The resultant homogenous blend was then compressed on a StylCam, a compaction simulator, simulating a Manesty Betapress operating at ˜37 RPM, into tablets with an individual tablet weight of 400 mg. 11.28 mm flat faced tooling (TSM-B) was used at compression forces reported.

### Control Directly Compressed Acetaminophen (APAP) Tablets (CE. 5A and CE.5B)

The comparative or control directly compressed tablet formulation (CE.5A) was prepared in the same manner as described in Example 5 using the ingredients in weight proportions listed in Table 4. In this example, the crosslinked PVP, fumed silica and Sodium Stearyl Fumarate were blended together with-out co-processing the same. The physical blend thus obtained was mixed with APAP and was compressed into tablets in the same manner as described in Example 5. Another comparative or control directly compressed APAP tablet formulation (CE.5B) was also prepared using only cross-linked PVP.

TABLE 4
Tablet Composition

| | Ex. 5A | Ex. 5B | Ex. 5C | CE. 5A | CE. 5B |
|---|---|---|---|---|---|
| Ingredients | (% w/w) | (% w/w) | (% w/w) | (% w/w) | (% w/w) |
| Acetamin- | 50 | 50 | 50 | 50 | 50 |
| ophen | | | | | |
| (APAP) DC | | | | | |
| Fast Flow | 30 | 30 | 30 | 30 | 30 |
| Lactose | | | | | |
| Cross-linked | Nil | Nil | Nil | 19.66 | 20 |
| PVP | | | | | |
| Co- | 20 | Nil | Nil | Nil | Nil |
| processed | | | | | |
| Excipient | | | | | |
| (Ex. 2 or 3B) | | | | | |
| Co- | Nil | 20 | Nil | Nil | Nil |
| processed | | | | | |
| Excipient | | | | | |
| (Ex. 3C) | | | | | |
| Co- | Nil | Nil | 20 | Nil | Nil |
| processed | | | | | |
| Excipient | | | | | |
| (Ex. 3A) | | | | | |
| Fumed Silica | — | — | — | 0.14 | — |
| Sodium | — | — | — | 0.2 | — |
| Stearyl | | | | | |
| Fumarate | | | | | |
| Total | 100.0 | 100.0 | 100.0 | 100.0 | 100.0 |

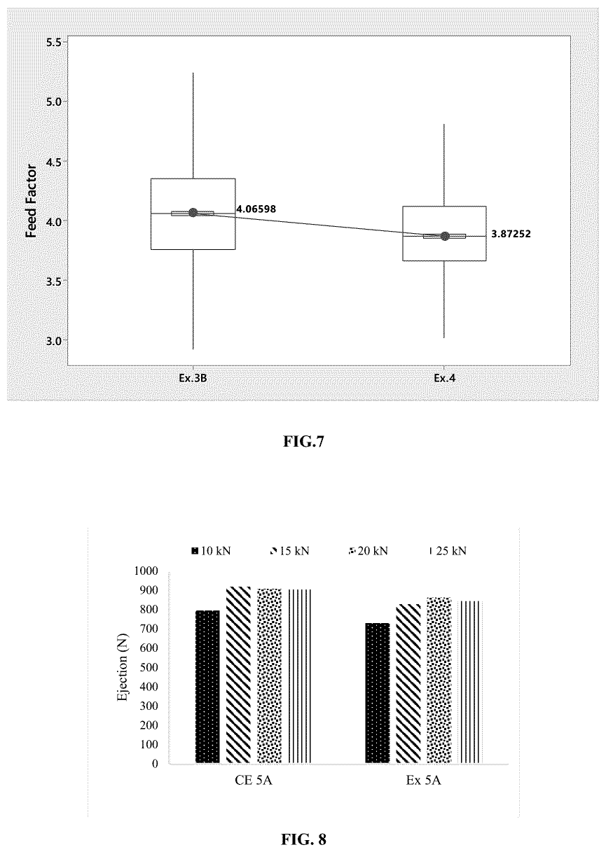

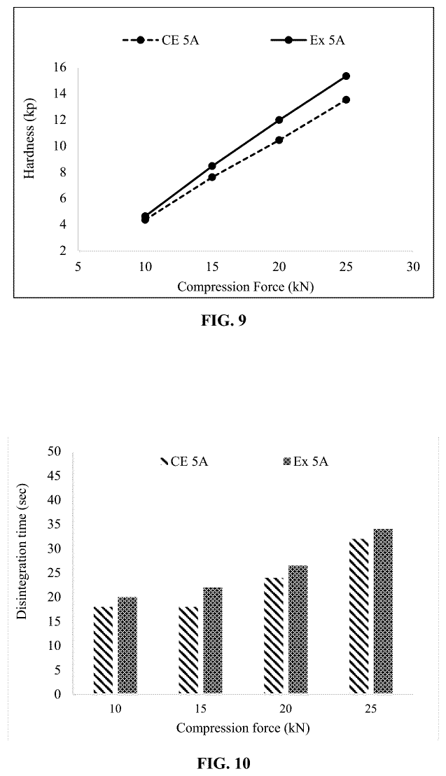

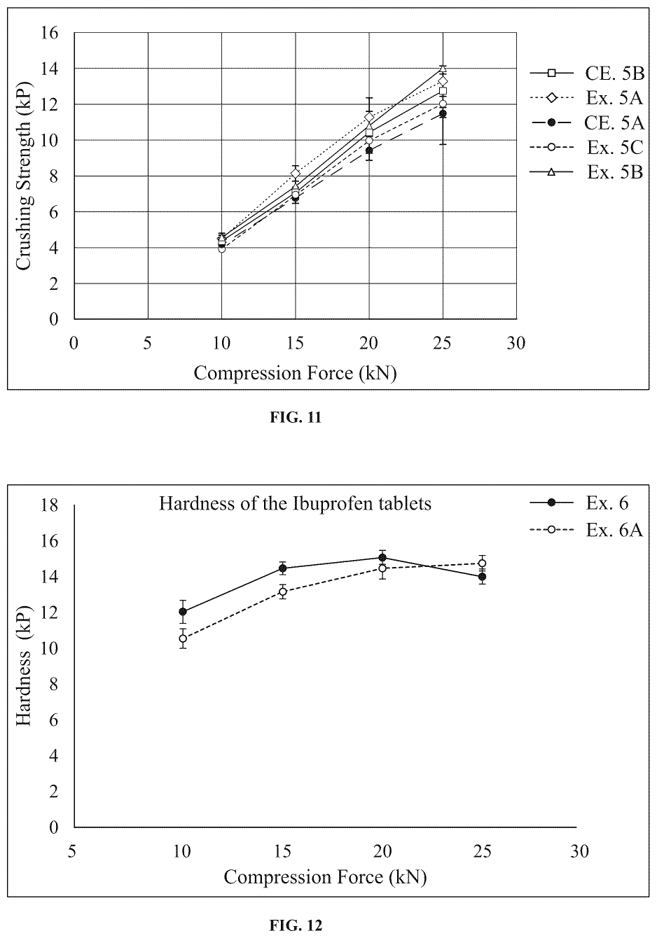

The ejection force data for the tablet samples, Ex.5A and CE.5A was recorded by the instrument and illustrated in FIG. 8. Tablets were retained and subsequently characterized for hardness and disintegration time. FIG. 9 and FIG. 10 further illustrate hardness (kp) and disintegration time (sec) of the tablet samples of Ex.5A and CE.5A. From the provided figures, it is evident that directly compressed tablet of Ex.5A achieves lower ejection force compared to the control tablet sample of CE.5A. Further, the directly compressed tablet of Ex.5A achieves higher tablet hardness and comparative tablet disintegration time as compared with the control tablet sample of CE.5A. FIG. 11 represents crushing strength (kP) of the present tablet samples i.e., EX.5A, EX.5B and EX.5C and its comparison with the control tablet samples CE.5A and CE.5B.

### Example 6: Directly Compressed (DC) Ibuprofen Tablet

In this example, another directly compressed (DC) tablet formulation was prepared using Ibuprofen as an active pharmaceutical ingredient. The tablet formulation (500 mg individual tablet weight) was prepared in the same manner as described in Example 5 using the ingredients in weight proportions listed in Table 5. The tablet sample of this example was characterized for hardness and disintegration time similar to the tablet samples of Example 5. The hardness and disintegration time is further illustrated in FIG. 12.

TABLE 5
Tablet Composition

| | Ex. 6 | Ex. 6A |
|---|---|---|
| Ingredients | (% w/w) | (% w/w) |
| Ibuprofen | 70 | 70 |
| Fast Flow Lactose | 17 | 17 |
| Klucel EXF (Hydroxypropyl cellulose) | 3 | 3 |
| Co-processed Excipient (Ex. 2) | 10 | Nil |
| Co-processed Excipient (Ex. 4) | Nil | 10 |
| Total | 100.00 | 100.00 |

What is claimed is:

1. A co-processed excipient comprising:
(i) from about 90.0 wt. % to about 99.9 wt. % of a vinyl lactam derived polymer comprising a monomer selected from the group consisting of N-vinyl-2-pyrrolidone, N-vinyl-2-caprolactam, N-vinyl-3-methyl-2-pyrrolidone, N-vinyl-3-methyl-2-caprolactam, N-vinyl-4-methyl-2-pyrrolidone, N-vinyl-4-methyl-2-caprolactam, N-vinyl-5-methyl-2-pyrrolidone, N-vinyl-5,5-dimethyl-2-pyrrolidone, N-vinyl-3,3,5-trimethyl-2-pyrrolidone, N-vinyl-5-methyl-5-ethyl-2-pyrrolidone, N-vinyl-3,4,5-trimethyl-3-ethyl-2-pyrrolidone, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, and combinations thereof;
(ii) from about 0.1 wt. % to about 5.0 wt. % of silica; and
(iii) from about 0.1 wt. % to about 5.0 wt. % of at least one lubricant.

2. The co-processed excipient of claim 1, wherein the vinyl lactam derived polymer is selected from the group consisting of polyvinylpyrrolidone, cross-linked polyvinyl pyrrolidone, and combinations thereof.

3. The co-processed excipient of claim 1, wherein the vinyl lactam derived polymer is present in an amount of from about 98 wt. % to about 99 wt. %, based on the total weight of the excipient composition.

4. The co-processed excipient of claim 1, wherein the silica is present in an amount of from about 0.5 wt. % to about 2.0 wt. %, based on the total weight of the excipient composition.

5. The co-processed excipient of claim 1, wherein the silica is selected from the group consisting of colloidal silica, fumed silica, a silicon dioxide, a calcium silicate and any combinations thereof.

6. The co-processed excipient of claim 1, wherein the lubricant is present in an amount of from about 0.1 wt. % to about 3.0 wt. %, based on the total weight of the excipient composition.

7. The co-processed excipient of claim 1, wherein the lubricant is selected from the group consisting of sodium stearyl fumarate, magnesium stearate, stearic acid, glyceryl dibehenate, and any combinations thereof.

8. The co-processed excipient of claim 1, wherein the co-processed excipient has a Brookfield flow factor of 5 to 9.

9. The co-processed excipient of claim 1, wherein the co-processed excipient is further combined with an active or functional ingredient selected from the group consisting of a paint, a coating, a personal care ingredient, a detergent, a pharmaceutical, a nutraceutical, a ceramic, an insulator, a pet food animal food, a human food, an agricultural product, an adhesive, an electroplating ingredient, an ink, a dye, a paper ingredient, a catalytic convertor, an electronic, and combinations thereof.

10. A process for preparing the co-processed excipient of claim 1, wherein the process comprising the step of:
(i) blending a vinyl lactam derived polymer, silica and a lubricant to obtain a blend, and
(ii) milling the resultant blend of step (i) to obtain a co-processed excipient.

11. The process of claim 10, is a single step process or a two-step process.

12. The process of claim 10, wherein the process is a two-step process comprising the steps of:
(i) blending a vinyl lactam derived polymer and silica;
(ii) adding a lubricant to the blend of step (i) to obtain a blend; and
(iii) milling the resultant blend of step (ii) to obtain a co-processed excipient.

13. The process of claim 10, wherein the co-processed excipient has a Brookfield flow factor of 5 to 9.

14. A composition comprising the co-processed excipient of claim 1 for use in an industrial application selected from paints and coatings, personal care, detergents, pharmaceuticals, nutraceuticals, ceramics, insulators, pet food, animal food and human food, agricultural products, adhesives, electroplating, inks, dyes, papers, catalytic convertors and electronics.

15. The composition of claim 14, wherein the composition is used in pharmaceuticals.

16. The composition of claim 14, wherein the composition is formulated into an oral dosage form by dry granulation, wet granulation, roller compaction, direct compression or hot melt extrusion processing, wherein said oral dosage forms is prepared by a batch-process or by a continuous process.

17. A directly compressible pharmaceutical composition comprising:
(i) at least one active pharmaceutical ingredient;
(ii) the co-processed excipient of claim 1; and
(iii) optionally one or more pharmaceutical acceptable additives.

18. The directly compressible pharmaceutical composition of claim 17, wherein the composition is formulated into modified release, controlled release, sustained release, extended release, immediate release or soluble dosage forms, wherein the composition is prepared by a batch-process or by a continuous process.

19. A process of preparing the directly compressible pharmaceutical composition of claim 17 comprising the steps of:
(i) blending the active pharmaceutical ingredient, the co-processed excipient of claim 1, and optionally one or more additives; and
(ii) compressing the resulting mixture of step (i).

20. The process of claim 19, is a batch-process or a continuous process.
